SEQUENCE LISTING (4 sequences)

SEQ ID NO: 1 - protein, 175 aa, Homo Sapiens
MLPPMALPSVSWMLLSCLMLLSQVQGEEPQRELPSARIRCPKGSKAYGSHCYALFLSPKSWTDADLACQKRPSGNLVSVLSGAEGSFVSSLVKSIGNSYSYVWIGLHDPTQGTEPNGEGWEWSSSDVMNYFAWERNPSTISSPGHCASLSRSTAFLRWKDYNCNVRLPYVCKFTD

SEQ ID NO: 2 - protein, 26 aa, Homo Sapiens
YVWIGLHDPTQGTEPNGEGWEWSSSD

SEQ ID NO: 3 - protein, 15 aa, Homo Sapiens
IGLHDPTQGTEPNGE

SEQ ID NO: 4 - protein, 1002 aa, Homo Sapiens
GGGAGGGTCCCTTCCTCAGGGAGCACAGGAACTCTGAGACTCAGCAAGGGTGTCCTGGGAGGGCTCGGGGATGGGAGAGTACACAGATTCACAACTCATTCAGAACTGTAGAAGATGATGGATGTGACCAAGATCACTTTAGTCCTAGGGGACTAGAGAAGGAAAATGACATGAGGCAGTGGGGTATCTGTGTGTTCTCCCACTGACCACGCTTTCTTTAGTGACTCCTGATTGCCTCCTCAAGTCGCAGACACTATGCTGCCTCCCATGGCCCTGCCCAGTGTATCTTGGATGCTGCTTTCCTGCCTCATGCTGCTGTCTCAGGTTCAAGGTGAAGAACCCCAGAGGGAACTGCCCTCTGCACGGATCCGCTGTCCCAAAGGCTCCAAGGCCTATGGCTCCCACTGCTATGCCTTGTTTTTGTCACCAAAATCCTGGACAGATGCAGATCTGGCCTGCCAGAAGCGGCCCTCTGGAAACCTGGTGTCTGTGCTCAGTGGGGCTGAGGGATCCTTCGTGTCCTCCCTGGTGAAGAGCATTGGTAACAGCTACTCATACGTCTGGATTGGGCTCCATGACCCCACACAGGGCACCGAGCCCAATGGAGAAGGTTGGGAGTGGAGTAGCAGTGATGTGATGAATTACTTTGCATGGGAGAGAAATCCCTCCACCATCTCAAGCCCCGGCCACTGTGCGAGCCTGTCGAGAAGCACAGCATTTCTGAGGTGGAAAGATTATAACTGTAATGTGAGGTTACCCTATGTCTGCAAGTTCACTGACTAGTGCAGGAGGGAAGTCAGCAGCCTGTGTTTGGTGTGCAACTCATCATGGGCATGAGACCAGTGTGAGGACTCACCCTGGAAGAGAATATTCGCTTAATTCCCCCAACCTGACCACCTCATTCTTATCTTTCTTCTGTTTCTTCCTCCCCGCTGTCATTTCAGTCTCTTCATTTTGTCATACGGCCTAAGGCTTTAAAGAGCAATAAAATTTTTAGTCTGC

(19) United States
(12) Patent Application Publication
(10) Pub. No.: US 2003/0212000 A1
(43) Pub. Date: Nov. 13, 2003

(54) Immunoprotective methods for beta cell neogenesis

(75) Inventor: William P. Van Antwerp, Valencia, CA (US)

Correspondence Address: GATES & COOPER LLP, HOWARD HUGHES CENTER, 6701 CENTER DRIVE WEST, SUITE 1050, LOS ANGELES, CA, 90045, US

(73) Assignee: Medtronic MiniMed, Inc.

(21) Appl. No.: 10/434,906

(22) Filed: May 9, 2003

Related U.S. Application Data
(60) Provisional application No. 60/379,202, filed on May 9, 2002.

(51) Int. Cl. A61K 38/17; G01N 33/53; G01N 33/537; G01N 33/543

(52) U.S. Cl. 514/12; 435/7.92

(57) ABSTRACT

The invention is based on the disclosure provided herein that a biologically active fragment of pancreatitis associated polypeptide can be used to stimulate beta cell growth and at the same avoid and overcome the T-cell mediated autoimmune attack on the pancreas. Typical embodiments of the invention include methods of inhibiting the onset of Type I diabetes in a mammalian subject predisposed to Type I diabetes comprising administering to the subject a therapeutically effective amount of a pancreatitis associated polypeptide comprising the amino acid sequence IGLHDPTQGTEPNGE (SEQ ID NO: 3).

This application claims the benefit of U.S. provisional patent application serial No. 60/379,202, filed May 9, 2002. The entire content of this provisional patent application is incorporated herein by reference.

### BACKGROUND OF THE INVENTION

1. Field of the Invention

The present invention relates to the prevention and treatment of diseases associated with pancreatic dysfunction.

2. Background of the Invention

Of the millions of individuals who are afflicted with diabetes, a large portion of them suffer from Type I diabetes, a syndrome caused by a lack of insulin which results from the loss of function and/or the destruction of insulin producing beta cells that are found in the pancreatic islets. In non-diabetic individuals, the beta cells produce sufficient amounts of insulin, a polypeptide which functions to regulate the ability of various tissues to absorb glucose as well as to maintain a steady blood-glucose concentration. If the body is unable to produce sufficient amounts of insulin, a high concentration of glucose will remain in the blood, and organs will not receive the glucose needed to function properly.

While the common remedial approach to diabetes has been to administer insulin directly to the patient, other approaches need to be considered as part of the comprehensive management of this pathology. One such approach entails reconstituting the environment of the healthy pancreas, for example by regenerating the insulin producing beta cells of the pancreatic islets in order to augment the production of insulin. A variety of protein growth factors and related molecules have been considered for this purpose.

Islet neogenesis associated proteins that are involved in growth and development of pancreatic cells have been identified in a first step in the development therapeutic compositions for the treatment of diabetes. There is a need in the art, however, to identify specific polypeptides for use in specific therapeutic modalities that take into account the complex physiological processes and factors associated with the development and progression of diabetes, in particular, the autoimmunity that is associated with this disease. The invention described herein meets this need.

### SUMMARY OF THE PREFERRED EMBODIMENTS

Embodiments of the invention described herein provide methods for the treatment of diabetes via the promotion of pancreatic cell growth. Typical methods disclosed herein are directed to therapeutic techniques involving the administration of a biologically active fragment of the pancreatitis-associated protein (“PAP”) protein in order to stimulate pancreatic cell growth. Typically, the biologically active fragment of the PAP protein is a peptide comprising the 15 amino acid sequence shown in SEQ ID NO: 3. The methods of the invention are further designed to avoid and/or minimize the leukocyte mediated autoimmune attack on the pancreas.

An illustrative embodiment of the invention is a method of using a biologically active fragment of pancreatitis associated protein to facilitate the growth of a pancreatic cell in a mammal in a manner that simultaneously minimizes the immunostimulation of a leukocyte produced by the mammal that immunospecifically recognizes an epitope present on the full length pancreatitis associated protein. In particular, the method involves administering to the mammal a biologically active fragment of pancreatitis associated protein comprising the amino acid sequence IGLHDPTQGTEPNGE (SEQ ID NO: 3). In this method, the mammal receiving this biologically active fragment of pancreatitis associated protein is selected to have a leukocyte that immunospecifically recognizes an epitope present on the homologous pancreatitis associated protein comprising the sequence MLPPMALPSVSWMLLSCLMLLSQVQGEEPQRELPSARIRCPKGSKAYGSHCYALFLSPKSWT DADLACQKRPSGNLVSVLSGAEGSFVSSLVKSIGNSYSYVWIGLHDPTQGTEPNGEGWEWS SSDVMNYFAWERNPSTISSPGHCASLSRSTAFLRWKDYNCNVRLPYVCKFTD (SEQ ID NO: 1) that is not present on the biologically active fragment of pancreatitis associated protein. Consequently, the administration of the biologically active fragment of pancreatitis associated protein facilitates the growth of the pancreatic cell but does not immunospecifically stimulate the leukocyte to the same degree as would the administration of an equivalent amount of the homologous pancreatitis associated protein.

Another typical embodiment of the invention is a method of inhibiting the onset of Type I diabetes in a mammalian subject predisposed to Type I diabetes comprising administering to the subject a therapeutically effective amount of a PAP peptide comprising the amino acid sequence shown in SEQ ID NO: 3. In such methods the PAP peptide can be administered via any one of the variety of methods known in the art. In one embodiment of the invention, the PAP peptide is administered to the subject via a continuous infusion pump. Embodiments of the invention include those wherein the mammalian subject has at least one T lymphocyte that is reactive to the 175 amino acid PAP protein shown in SEQ ID NO: 1, yet is not reactive to the PAP peptide shown in SEQ ID NO: 3. Preferably the peptide formulation includes a pharmaceutically acceptable carrier such a physiologic saline. Optionally the PAP peptide is conjugated to a polyol. Related embodiments of the invention include administering a second agent (including those typically used in the treatment of diabetes) such as one selected from the group consisting of human insulin, human glucagon-like peptide 1 (GLP 1) and Fas ligand. While the methods disclosed herein are directed primarily to the treatment of type I diabetes, the use of such methods in the prevention and treatment of other diseases associated with pancreatic dysfunction are also contemplated.

A related embodiment of the invention is a method of stimulating the growth of pancreatic cells in an individual having a predisposition to Type I diabetes comprising administering to the subject a PAP peptide comprising the amino acid sequence shown in SEQ ID NO: 3. A predisposition to diabetes can be measured by any one of the wide variety of methods known in the art such as those described in chapter 7 of TYPE I DIABETES: MOLECULAR, CELLULAR AND CLINICAL IMMUNOLOGY (A. Pugliese and G. S. Eisenbarth eds.). In a typical embodiment, the predisposition to Type I diabetes is determined by the presence of islet cell autoantibodies or insulin autoantibodies. In a specific method, the islet cell autoantibody recognizes islet cell antigen 512 (ICA 512). Alternatively, the predisposition to Type I diabetes is determined by the presence of T cells that recognize an epitope on the 175 amino acid PAP protein shown in SEQ ID NO: 1.

Yet another embodiment of the invention is a method of inhibiting the onset of Type I diabetes in a mammalian subject predisposed to Type I diabetes comprising administering to the subject a therapeutically effective amount of a PAP peptide comprising the amino acid sequence shown in SEQ ID NO: 3, wherein the PAP peptide does not possess an epitope present in the PAP protein comprising the amino acid sequence shown in SEQ ID NO: 1 that is recognized by autoimmune T-cells in the individual.

Yet another embodiment of the invention is a method of stimulating neogenesis of pancreatic beta cells in an individual having a predisposition to Type I diabetes comprising determining whether the mammalian subject possesses a T lymphocyte that is reactive to the 175 amino acid PAP protein shown in SEQ ID NO: 1, is not reactive to the PAP peptide shown in SEQ ID NO: 3, and then administering to the subject a therapeutically effective amount of a PAP peptide comprising the amino acid sequence shown in SEQ ID NO: 3 to the subject.

Yet another embodiment of the invention is a method of inhibiting the expression in a mammalian subject of an epitope present on the 175 amino acid PAP protein shown in SEQ ID NO: 1, wherein the epitope is recognized by an autoimmune T cell in Type I diabetes, the method comprising exposing a pancreatic cell in the subject to an amount of a PAP peptide comprising the amino acid sequence shown in SEQ ID NO: 3 sufficient to stimulate pancreatic cell growth. Optionally, the expression of the 175 amino acid PAP protein shown in SEQ ID NO: 1 is measured via a northern blot analysis or polymerase chain reaction analysis. Optionally, the proliferative response of the pancreatic cell is measured via [³H]TdR incorporation. Alternatively, the promotion of pancreatic growth is measured via an assay that measures cell adhesion on PAP peptide. In such methods the subject typically exhibits at least one factor associated with a predisposition to Type I diabetes. Preferably the factor associated with a predisposition to Type I diabetes is determined is the presence of islet cell autoantibodies or insulin autoantibodies. In a preferred embodiment, the factor associated with a predisposition to Type I diabetes is determined is the presence of an autoantibody that recognizes islet cell antigen 512 (ICA 512). Alternatively the factor associated with a predisposition to Type I diabetes is determined is the presence of an autoimmune T-cell that recognizes an epitope present on the 175 amino acid PAP protein shown in SEQ ID NO: 1.

Yet another embodiment of the invention is a composition comprising a PAP peptide having the amino acid sequence shown in SEQ ID NO: 3 and a pharmaceutically acceptable carrier. Optionally the peptide is conjugated to a polyol and/or a heterologous polypeptide sequence. In one embodiment, such composition may be included in an article of manufacture or kit. The composition may be a pharmaceutically acceptable formulation useful, for instance, in inducing or stimulating neogenesis in mammalian beta cells or for treating an immune related disorder, such as diabetes. In one embodiment of the invention, the PAP peptide is a peptide comprising amino acids 104-118 and having an amino acid substitution at one or more amino acid residues.

### BRIEF DESCRIPTION OF THE DRAWINGS

FIGS. 1A and 1B show the PAP nucleotide and amino acid sequences respectively. PAP is further described in Christa et al., Am. J. Physiol. 271 (6 Pt 1), G993-G1002 (1996) and has GenBank Accession Nos. NM_{—}138938 and NP_{—}620355.

### DETAILED DESCRIPTION OF THE PREFERRED EMBODIMENTS

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art, such as, for example, the widely utilized molecular cloning methodologies described in see Ausubel et al., Current Protocols in Molecular Biology, Wiley Interscience Publishers, (1995) and Sambrook et al., Molecular Cloning: A Laboratory Manual 2nd. edition (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

A. Brief Characterization of Aspects of the Invention

Embodiments of the invention described herein provide methods for the treatment of pancreatic dysfunction via the promotion of pancreatic growth and/or rehabilitation via processes such as islet neogenesis. Typical methods are directed to such therapeutic techniques including as the administration of a PAP peptide having the ammo acid sequence shown in SEQ ID NO: 3 to an individual having a predisposition to diabetes in order to facilitate pancreatic growth. These methods of the invention are designed to avoid and/or overcome the leukocyte mediated autoimmune attack on the pancreas. The sections below disclose exemplary methods for identifying individuals predisposed to Type I diabetes, methods for using PAP polypeptides and polynucleotides of the invention and typical embodiments of the invention.

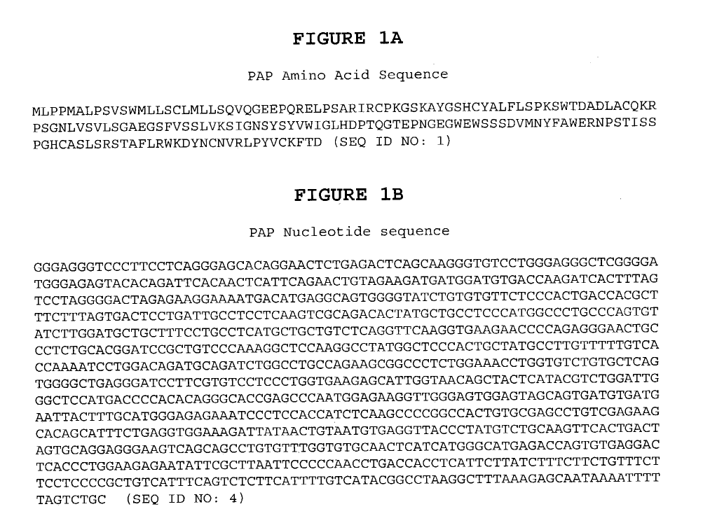

The terms “pancreatitis-associated protein (PAP)” as used herein refers to a protein encoded by the gene that encodes SEQ ID NO: 1. This gene is described in Christa et al., Am. J. Physiol. 271 (6 Pt 1), G993-G1002 (1996) and has GenBank Accession Nos. NM_{—}138938 and NP_{—}620355. A PAP polypeptide can include amino acid residues 1-175 of the ammo acid sequence shown in FIG. 1 (SEQ ID NO:1), the post translationally processed form of 1-175 of the amino acid sequence shown in FIG. 1 (e.g. the protein produced after cleavage of a leader peptide that occurs as part of a maturation process), as well as biologically active fragments, deletional, insertional, or substitutional variants of the above sequences. The PAP polypeptides of the invention may be encoded by the native nucleotide sequence shown in Table 1 (SEQ ID NO:4). In other embodiments, the fragments or variants are biologically active and have at least about 80% amino acid sequence identity, more preferably at least about 90% sequence identity, and even more preferably, at least 95%, 96%, 97%, 98%, or ⁹⁹% sequence identity with any one of the above recited PAP sequences. In certain embodiments, the fragment has at least about 66%, ⁷³%, 80%, 86% or 93% amino acid sequence identity with the amino acid sequence shown in SEQ ID NO:3. Optionally, the PAP polypeptide is encoded by a nucleotide sequence which hybridizes under stringent conditions with the encoding polynucleotide sequence provided in FIG. 1 (SEQ ID NO:4).

“Stringent conditions”, as defined herein, are identified by those that: (1) employ low ionic strength and high temperature for washing; 0.015 M sodium chloride/0.0015 M sodium citrate/0.1% sodium dodecyl sulfate at 50° C.; (2) employ during hybridization a denaturing agent; 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

B. Methods for Identifying Individuals Predisposed to Type I Diabetes

Embodiments of the invention disclosed herein include methods of administering PAP peptide to an individual identified as having diabetes or a predisposed susceptibility to diabetes. The term “administer” means to introduce formulation of the present invention into the body of a patient in need thereof to treat a disease or condition. In such methods, one can evaluate an individual for a predisposed susceptibility to diabetes by examining an individual for the presence or absence of one of the various factors that are known in the art to be associated with such a predisposition. In this context, many genetic loci associated with a predisposition to diabetes have been identified including for example those within the human leukocyte antigen (HLA) region on the short arm of chromosome 6, loci which account for up to 50% of the inheritable diabetes risk. A review of illustrative susceptibility loci can be found in chapter 7 of “Type I diabetes: MOLECULAR, CELLULAR, AND CLINICAL IMMUNOLOGY”; by A Pugliese and G. S. Eisenbarth which is incorporated herein by reference. As illustrated in this text, a variety of methods for examining and characterizing such susceptibility are known in the art. Related methods examining other loci are known in the art, as disclosed for example in U.S. Pat. No. 6,162,604 which teaches the methods for examining a predisposition to pathologies such as diabetes by observing the differential expression of genetic markers associated with programmed cell death in individuals having autoimmune syndromes such as Type I diabetes. Another embodiment of such methods is described in U.S. Pat. No. 6,187,533 which teaches that that an analysis of mutations in the HNF1α, HNF1β and HNF4α genes can be diagnostic for diabetes.

In addition to susceptibility loci, additional factors associated with Type I diabetes include the presence of autoantibodies and autoimmune T cells which can be identified in of individuals prior to onset. Autoantibodies and include islet cell autoantibodies of the cytoplasmic type (ICA) (Bottazzo et al., (1974) Lancet. 2:1279-1283); islet cell surface autoantibodies (ICSA) (Maclaren et al. (1975) Lancet. i:997-1000); insulin autoantibodies (IAA) (Palmer et al., (1983) Science 222:1337-1339) and the possible antidiotypic insulin receptor autoantibodies (Ins.R. A.) (Maron et al. (1983) Nature 303:817-818). A number of commercially available tests for such antibodies are known in the art, for example the assay for anti-islet cell antigen 512 (ICA 512) autoantibody produced by Quest Diagnostics™. In addition, a variety of methods for determining the presence of autoreactive T cells are known in the art (see, e.g. Roep et al., Journal of Autoimmunity 13, 267-283 (1999) and Gurr et al., Diabetes 51: 339-346, 2002 which are incorporated herein by reference).

A typical embodiment of a process for the early detection of insulin dependent diabetes (IDD) before clinical symptoms appear (e.g. the need to modify dietary intake and/or the need for exogenous insulin) via the detection, in a sample of biological fluid, of an autoantibody which is highly specific to individuals who will later develop the clinical manifestations of IDD. For example, as described in U.S. Pat. No. 6,300,089, autoantibodies to an islet cell 64,000 Mr protein, are present up to several years before the clinical manifestations of IDD are observed. Alternatively one can examine an individual for a factor associated with a predisposition to diabetes by looking for the presence of autoimmune T-cell clones associated with this pathology. For example, one can examine the T cell recognition of antigenic determinants on insulin or PAP using one of the methods typically used in the art for this purpose (see, e.g. Nell et al., J Clin Invest 1985 76(6):2070-2077; and Dotta et al., Eur J Endocrinol 1999, 141(3):272-278).

C. Methods Using PAP Polypeptides and Polynucleotides of the Invention

The methods disclosed herein may be employed in protocols for inhibiting the onset of Type I & II diabetes as well as in related protocols involving the treatment of associated pathological conditions in mammals such as prediabetic pancreatic dysfunction. In typical methods, a PAP polypeptide is administered to a mammalian subject, alone or in combination with still other therapeutic agents or techniques. Diagnosis in mammals of the various pathological conditions described herein such as diabetes or predisposition to diabetes can be made by the skilled practitioner using one or more of the diagnostic techniques known in the art including those described above. In addition, the relevant art teaches a number of criteria for the characterization of prediabetic conditions prior to as well as associated with onset of Type I diabetes (see, e.g. Mrena et al., Pediatrics 1999, 104 (4 Pt 1): 925-30; Tillil et al., Z Arztl Fortbild Qualitatssich 1998; 92(7):456-466; DeAizpurua et al., J Autoimmun 1992, 5(6):759-770; Lo et al., Diabetalogia 1992, 35(3):277-282; Kuglin et al., Diabet Med 1990, 7(4):310-314; Greenberg et al., Clin Chin Acta 2002, 315(1-2):61-69; Kimpimaki et al., J Pediatr Endocrinol Metab 2001, 14 Suppl 1:575-587; and Ziegler et al., Diabetes Care 1990, 13(7):762-765).

Polypeptides useful in the methods of the invention encompass both naturally occurring PAP polypeptides as well as variations and modified forms thereof. “PAP polypeptide or protein” includes naturally occurring mammalian PAPs, and biologically active variants and fragments thereof. The 175 amino acid sequence of human PAP (SEQ ID NO: 1, also known as PAP-H or HIP) is shown in Table 1 below (see, e.g. Orelle et al., J. Clin. Invest. 90 (6), 2284-2291 (1992); GenBank Accession No. AAB24642). A preferred PAP polypeptide is a biologically active fragment of the 175 amino acid sequence of human PAP comprising amino acids 100-125 which is shown in Table 1 below (SEQ ID NO: 2). A highly preferred PAP polypeptide is a biologically active fragment of the 175 amino acid sequence of human PAP comprising amino acids 104-118 which is shown in Table 1 below (SEQ ID NO: 3). The PAP polypeptides used in the methods disclosed herein typically exhibit the biological activity found in the fragment comprising amino acids 104-118.

As used herein, the term “polypeptide” means a polymer of at least about 4, 5, 6, 7, or 8 or more amino acids. This term is often used interchangeably with “protein”. As used herein, the term “peptide” as in “PAP peptide” means a polypeptide having about 25 amino acids or less. In this context, a PAP peptide of 25 amino acids or less can be linked to a heterologous polypeptide sequence to form a polypeptide that is larger than 25 amino acids and has the PAP peptide embedded within it (e.g. a fusion protein comprising the PAP sequence shown in SEQ D NO: 3 and portions of an immunoglobulin polypeptide sequence). Throughout the specification, standard three letter or single letter designations for amino acids are used.

The biological activity of various PAP polypeptides can be examined using one of the assays for characterizing PAP and/or pancreatic and/or beta cell function known in the art (see, e.g. Vinik et al., Horm Metab Res June 1997;29(6):278-93; Rafaeloff et al., J. Clin. Invest. 1997, 99:2100-2109; Watanabe et al., PNAS 1994, 91:3589-3592; Del Zotto et al., J Endocrinol June 2000;165(3):725-33; Vinik et al., Tumour Biol 1993;14(3):184-200; Hill et al., Diabetes Care 1998;21 Suppl 2:B60-9 Xu et al., Diabetes December 1999;48(12):2270-6: Yamamoto et al., Diabetes December 2000;49(12):2021-7; Su et al., Pancreas, 1999, 19(3):239-247; Heller et al., Anesthesiology, 1999, 91:1408-1414; Rosenberg, Microscopy Research and technique, 1998, 43:337-346 and Del Zotto et al., Diabetes Metab Res Rev March-April 1999;15(2):106-12). Additional methods that can be used for examining PAP and related molecules are described in U.S. Pat. Nos. 5,840,531, 5,804,421, 5,834,590, 5,436,169 and 5,935,813. Typical assay for examining PAP activity are shown in Examples 1, 2 and 3 below.

PAP polypeptides for use in the methods disclosed herein can be PAP variants, PAP fragments, analogues, and derivatives. By “analogues” is intended analogues of either PAP or a PAP fragment that comprise a native PAP sequence and structure, having one or more amino acid substitutions, insertions, or deletions. Peptides having one; or more peptoids (peptide nimics) are also encompassed by the term analogues (WO 91/04282). By “derivatives” is intended any suitable modification of PAP, PAP fragments, or their respective analogues, such as glycosylation, phosphorylation, or other addition of foreign moieties (e.g. pegylation as described below), so long as the desired activity is retained. Methods for masking PAP fragments, analogues, and derivatives are available in the art.

As used herein, the PAP gene and PAP polypeptide includes the rodent and human PAP genes and polypeptides specifically described herein, as well as biologically active structurally and/or functionally similar variants or homologs of the foregoing. PAP peptide homologs generally share at least about 90%, 95% or more amino acid homology to the corresponding PAP wild type sequences such as those shown in SEQ ID NOs: 1, 2 or 3 (using BLAST criteria). For example, % identity values may be generated by WU-BLAST-2 (Altschul et al., 1996, Methods in Enzymology 266:460-480; http://blast.wusd/edu/blast/README.html). PAP nucleotide homologs preferably share 50%, 60%, 70%, 80%, 90% or more nucleic acid homology (using BLAST criteria). In some embodiments, however, lower homology is preferred so as to select preferred residues in view of species-specific codon preferences and/or optimal peptide epitopes tailored to a particular target population, as is appreciated by those skilled in the art.

Shorter PAP peptide compounds of this invention can be made by chemical synthesis or by employing recombinant technology. These methods are known in the art. Chemical synthesis, especially solid phase synthesis, is preferred for short (e.g., less than 50 residues) peptides or those containing unnatural or unusual amino acids such as D-Tyr, Orithine, amino adipic acid, and the like. Recombinant procedures are preferred for longer polypeptides. When recombinant procedures are selected, a synthetic gene may be constructed de novo or a natural gene may be mutated by, for example, cassette mutagenesis. Set forth below are exemplary general recombinant procedures.

Optionally, a PAP peptide can be produced using methods known in the art such as recombinant DNA techniques. The DNA techniques contemplate, in simplified form, taking the gene, either natural or synthetic, encoding the peptide; insetting it into an appropriate vector; inserting the vector into an appropriate host cell; culturing the host cell to cause expression of the gene; and recovering or isolating the peptide produced thereby. Preferably, the recovered peptide is then purified to a suitable degree.

Somewhat more particularly, the DNA sequence encoding a PAP peptide is cloned and manipulated so that it may be expressed in a convenient host. DNA encoding parent polypeptides can be obtained from a genomic library, from cDNA derived from mRNA from cells expressing the peptide, or by synthetically constructing the DNA sequence (Sambrook et al., Molecular Cloning: A Laboratory Manual (2d ed.), Cold Spring Harbor Laboratory, N.Y., 1989). The parent DNA is then inserted into an appropriate plasmid or vector which is used to transform a host cell. In general, plasmid vectors containing replication and control sequences which are derived from species compatible with the host cell are used in connection with those hosts. The vector ordinarily carries a replication site, as well as sequences which encode proteins or peptides that are capable of providing phenotypic selection in transformed cells. For example, E. coli may be transformed using pBR322, a plasmid derived from an E. coli species. Mandel et al., J. Mol. Biol. 53: 154 (1970). Plasmid pBR322 contains genes for ampicillin and tetracycline resistance, and thus provides easy means for selection. Other vectors include different features such as different promoters, which are often important in expression. For example, plasmids pKK223-3, pDR720, and pPL-lambda represent expression vectors with the tac, trp, or P_{L} promoters that are currently available (Pharmacia Biotechnology).

Other preferred vectors can be constructed using standard techniques by combining the relevant traits of the vectors described above. Relevant traits include the promoter, the ribosome binding site, the decorsin or ornatin gene or gene fusion (the Z domain of protein A and decorsin or ornatin and its linker), the antibiotic resistance markers, and the appropriate origins of replication.

The host cell may be prokaryotic or eukaryotic. Prokaryotes are preferred for cloning and expressing DNA sequences to produce parent polypeptide, segment-substituted peptides, residue-substituted peptides, and peptide variants. For example, E. coli K12 strain 294 (ATCC No. 31446) may be used as well as E. coli B, E. coli X1776 (ATCC No. 31537), and E. coli c600 and c600hfl, E. coli W3110 (F-, gamma-, prototrophic/ATCC No. 27325), bacilli such as Bacillus subtilis, and other enterobacteriaceae such as Salmonella typhimurium or Serratia marcesans, and various Pseudomonas species. The preferred prokaryote is E. coli W3110 (ATCC 27325). When expressed by prokaryotes the peptides typically contain an N-terminal methionine or a formyl methionine and are not glycosylated. In the case of fusion proteins, the N-terminal methionine or formyl methionine resides on the amino terminus of the fusion protein or the signal sequence of the fusion protein. These examples are, of course, intended to be illustrative rather than limiting.

In addition to prokaryotes, eukaryotic organisms, such as yeast cultures, or cells derived from multicellular organisms may be used. In principle, any such cell culture is workable. However, interest has been greatest in vertebrate cells, and propagation of vertebrate cells in culture (tissue culture) has become a reproducible procedure. Tissue Culture, Academic Press, Kruse and Patterson, editors (1973). Examples of such useful host cell lines are VERO and HeLa cells, Chinese Hamster Ovary (CHO) cell lines, W138, 293, BHIK, COS-7 and MDCK cell lines.

Molecules comprising fusion proteins that combine a PAP peptide and a heterologous polypeptide are also included within the scope of the invention. Illustrative embodiments of the invention are gene fusions, wherein the gene encoding the desired peptide (e.g. PAP) is associated, in the vector, with a gene encoding another protein or a fragment of another protein. This results in the desired peptide being produced by the host cell as a fusion with another protein or peptide. The “other” protein or peptide is often a protein or peptide which can be secreted by a bacterial or mammalian cell, making it possible to isolate and purify the desired peptide from the culture medium and eliminating the necessity of destroying the host cells which arises when the desired peptide remains inside the cell. Alternatively, the fusion protein can be expressed intracellularly. It is useful to use fusion proteins that are highly expressed. In a preferred embodiment, a PAP peptide can be fused with an immunoglobulin or a particular region of an immunoglobulin such as the Fc region of an IgG molecule. Such fusions can be generated for example in order to extend the in vivo plasma half-life of a desired peptide composition. In a typical embodiment, the immunoglobulin fusion includes the hinge, CH2 and CH3, or the hinge, CHI, CH2 and CH3 regions of an IgGI molecule. For the production of immunoglobulin fusions see, e.g., U.S. Pat. No. 5,428,130 which is incorporated herein by reference.

The term “variant” refers to a molecule that exhibits a variation from a described type or norm, such as a polypeptide that has one or more different amino acid residues in the corresponding position(s) of a specifically described polypeptide. An analog is an example of a variant polypeptide. As used herein, the PAP-related gene and PAP-related polypeptide includes the PAP genes and polypeptides specifically described herein, as well as structurally and/or functionally similar variants or analog of the foregoing. PAP peptide analogs generally share at least about 50%, 60%, 70%, 80%, 90% or more amino acid homology (using BLAST criteria). PAP nucleotide analogs preferably share 50%, 60%, 70%, 80%, 90% or more nucleic acid homology (using BLAST criteria). In some embodiments, however, lower homology is preferred so as to select preferred residues in view of species-specific codon preferences and/or optimal peptide epitopes tailored to a particular target population, as is appreciated by those skilled in the art.

Embodiments of the invention disclosed herein include a wide variety of art-accepted variants or analogs of PAP polypeptides such as polypeptides having amino acid insertions, deletions and substitutions. PAP peptide variants can be made, for example, by substituting amino acids during solid phase peptide synthesis. PAP variants can also be made using recombinant DNA methods known in the art such as site-directed mutagenesis, alanine scanning, and PCR mutagenesis. Site-directed mutagenesis (Carter et al., Nucl. Acids Res., 13:4331 (1986); Zoller et al., Nucl. Acids Res., 10:6487 (1987)), cassette mutagenesis (Wells et al., Gene, 34:315 (1985)), restriction selection mutagenesis (Wells et al., Philos. Trans. R. Soc. London SerA, 317:415 (1986)) or other known techniques can be performed on the cloned DNA to produce the PAP variant DNA. Resulting mutants can be tested for biological activity. Sites critical for binding can be determined by structural analysis such as crystallization, photoaffinity labeling, or nuclear magnetic resonance. See, deVos et al. (1992) Science 255:306 and Smith et al. (1992:) J. Mol. Biol. 224:899.

As is known in the art, amino acid substitutions can frequently be made in a polypeptide without compromising the desired functional activity of the polypeptide. Consequently polypeptides of the invention can comprise such substitutions. Such changes can include conservative substitutions such as occurs by substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the polypeptide. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments.

In making amino acid substitutions, scanning amino acid analysis can be employed to identify one or more amino acids along a contiguous sequence that is involved in a specific biological activity such as a protein-protein interaction. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant. Alanine is also typically preferred because it is the most common amino acid. Further, it is frequently found in both buried and exposed positions (Creighton, The Proteins, (W. H. Freeman & Co., N.Y.); Chothia, J. Mol. Biol., 150:1 (1976)). If alanine substitution does not yield adequate amounts of variant, an isostetic amino acid can be used.

An illustrative embodiment of a PAP variant polypeptide of the present invention is a peptide comprising amino acids 104-118 of PAP shown in SEQ ID NO: 1 which is shown in Table 1 below (SEQ ID NO: 3), wherein the peptide has an amino acid substitution at one or more amino acid residues of residues 104-118. For example, I at position 104 can be replaced with A, C, D, E, F, G, H, K, L, M, N, P, Q, R, S, T, V, W, or Y; G at position 105 can be replaced with A, C, D, E, F, H, I, K, L, M, N, P, Q, R, S, T, V, W, or Y; L at position 106 can be replaced with A, C, D, E, F, G, H, I, K, M, N, P, Q, R, S, T, V, W, or Y; H at position 107 can be replaced with A, C, D, E, F, G, I, K, L, M, N, P, Q, R, S, T, V, W, or Y; D at position 108 can be replaced with A, C, E, F, G, H, I, K, L, M, N, P, Q, R, S, T, V, W, or Y; P at position 109 can be replaced with A, C, D, E, F, G, H, I, K, L, M, N, Q, R, S, T, V, W, or Y; T at position 110 can be replaced with A, C, D, E, F, G, H, I, K, L, M, N, P, Q, R, S, V, W, or Y; Q at position 111 can be replaced with A, C, D, E, F, G, H, I, K, L, M, N, P, R, S, T, V, W, or Y; G at position 112 can be replaced with A, C, D, E, F, H, I, K, L, M, N, P, Q, R, S, T, V, W, or Y; T at position 113 can be replaced with A, C, D, E, F, G, H, I, K, L, M, N, P, Q, R, S, V, W, or Y; E at position 114 can be replaced with A, C, D, F, G, H, I, K, L, M, N, P, Q, R, S, T, V, W, or Y; P at position 115 can be replaced with A, C, D, E, F, G, H, I, K, L, M, N, Q, R, S, T, V, W, or Y; N at position 116 can be replaced with A, C, D, E, F, G, H, I, K, L, M, P, Q, R, S, T, V, W, or Y; G at position 117 can be replaced with A, C, D, E, F, H, I, K, L, M, N, P, Q, R, S, T, V, W, or Y; and E at position 118 can be replaced with A, C, D, F, G, H, I, K, L, M, N, P, Q, R, S, T, V, W, or Y. Related embodiments of such peptides include those having amino acid substitutions at 2, 3, 4, 5, 6, 7, 8, 9, or 10 amino acid residues of residues 104-118.

Variant PAP polypeptides and PAP peptides useful in the methods of the present invention possess PAP biological activity as can be determined, for example by an assay such as those described herein. Specifically, they must possess the desired biological activity of the native polypeptide, for example the ability to promote the growth of pancreatic beta cells as described in Examples 1 and 2 below. For the purposes of the invention, a “PAP variant” will exhibit at least about 10% of the activity of the PAP shown in SEQ ID NO: 1, SEQ ID NO: 2 or SEQ ID NO: 3. More typically, variants exhibit more than about 50% of this activity; even more typically, variants exhibit more than about 90% of this activity.

The description below also provides methods of producing PAP covalently attached (hereinafter “conjugated”) to one or mote chemical groups. Chemical groups suitable for use in a PAP conjugate of the present invention are preferably not significantly toxic or immunogenic. The chemical group is optionally selected to produce a PAP conjugate that can be stored and used under conditions suitable for storage. A variety of exemplary chemical groups that can be conjugated to polypeptides are known in the art and include for example carbohydrates, such as those carbohydrates that occur naturally on glycoproteins, and non-proteinaceous polymers, such as polyols (see, e.g., U.S. Pat. No. 6,245,901).

A polyol, for example, can be conjugated to polypeptides such as the PAP peptide shown in SEQ ID NO: 3 at one or more amino acid residues, as is disclosed in WO 93/00109. The polyol employed can be any water-soluble poly(alkylene oxide) polymer and can have a linear or branched chain. Suitable polyols include those substituted at one or more hydroxyl positions with a chemical group, such as an alkyl group having between one and four carbons. Typically, the polyol is a poly(alkylene glycol), such as poly(ethylene glycol) (PEG), and thus, for ease of description, the remainder of the discussion relates to an exemplary embodiment wherein the polyol employed is PEG and the process of conjugating the polyol to a polypeptide is termed “pegylation.” However, those skilled in the art recognize that other polyols, such as, for example, poly(propylene glycol) and polyethylene-polypropylene glycol copolymers, can be employed using the techniques for conjugation described herein for PEG. Illustrative examples of cytokines conjugated with PEG are shown, for example, in U.S. Pat. No. 5,795,569; U.S. Pat. No. 4,902,502; Wang et al., Biochemistry 2000, 39, 10634-10640; Leong et al., Cytokine 2001, 16(3): 24-36; and Kozlowski et al., BioDrugs 2001; 15(7): 419-429.

While the residues may be any reactive amino acids on the protein, such as the N-terminal amino acid group, one reactive amino acid is cysteine, which is linked to the reactive group of the activated polymer through its free thiol group as shown, for example, in WO 99/03887, WO 94/12219, WO 94/22466, U.S. Pat. No. 5,766,897, U.S. Pat. No. 5,206,344, U.S. Pat. No. 5,166,322, and U.S. Pat. No. 5,206,344. Alternatively the reactive group is lysine, which is linked to the reactive group of the activated polymer through its free epsilon-amino group, or glutamic or aspartic acid, which is linked to the polymer through an amide bond. This reactive group can then react with, for example, the α and ε amines of proteins to form a covalent bond. Conveniently, the other end of the PEG molecule can be “blocked” with a non-reactive chemical group, such as a methoxy group, to reduce the formation of PEG-crosslinked complexes of protein molecules.

A variety of methods for pegylating polypeptides are known in the art. Specific methods of producing polypeptides conjugated to PEG include the methods described in U.S. Pat. No. 4,179,337, U.S. Pat. No. 4,935,465, U.S. Pat. No. 5,824,784 and U.S. Pat. No. 5,849,535. Typically the polypeptide is covalently bonded via one or more of the ammo acid residues of the polypeptide to a terminal reactive group on the polymer, depending mainly on the reaction conditions, the molecular weight of the polymer, etc. The polymer with the reactive group(s) is designated herein as activated polymer. The reactive group selectively reacts with free amino or other reactive groups on the polypeptide. The PEG polymer can be coupled to the amino or other reactive group on the polypeptide in either a random or a site specific manner. It will be understood, however, that the type and amount of the reactive group chosen, as well as the type of polymer employed, to obtain optimum results, will depend on the particular polypeptide or polypeptide variant employed to avoid having the reactive group react with too many particularly active groups on the polypeptide.

The degree of pegylation of PAP of the present invention can be adjusted to provide a desirably increased in amino half-life (hereinafter “half-life”), compared to the corresponding non-pegylated PAP. It is believed that the half-life of a pegylated PAP typically increases incrementally with increasing degree of pegylation. The degree and sites of pegylation of a polypeptide are determined, e.g., by (1) the number and reactivities of pegylation sites (e.g., primary amines) and (2) pegylation reaction conditions.

Pegylated polypeptides can be characterized by SDS-PAGE, gel filtration, NMR, peptide mapping, liquid chromatography-mass spectrophotometry, and in vitro biological assays. The extent of pegylation is typically first shown by SDS-PAGE. Polyacrylamide gel electrophoresis in 10% SDS is typically run in 10 mM Tris-HCl pH 8.0, 100 mM NaCl as elution buffer. To demonstrate which residue is pegylated, peptide mapping using proteases such as trypsin and Lys-C protease can be performed. Thus, samples of pegylated and non-pegylated peptides can be digested with a protease such as Lys-C protease and the resulting peptides separated by a technique such as reverse phase HPLC. The chromatographic pattern of peptides produced can be compared to a peptide map previously determined for the polypeptide. Each peak can then be analyzed by mass spectrometry to verify the size of the fragment in the peak. The fragment(s) that carried PEG groups are usually not retained on the HPLC column after injection and disappear from the chromatograph. Such disappearance from the chromatograph is an indication of pegylation on that particular fragment that should contain at least one pegylatable amino acid residue.

The PAP may be administered directly by introducing a PAP polypeptide, PAP variant or PAP fragment into or onto the subject. Alternatively, the PAP may be produced in situ following the administration of a polynucleotide encoding a PAP polypeptide, PAP variant or PAP fragment may be introduced into the subject.

Polynucleotides for use in the methods disclosed herein may be naturally occurring, such as allelic variants, homologs, orthologs, or may be constructed by recombinant DNA methods or by chemical synthesis. Alternatively, the variant polypeptides may be non-naturally occurring and made by techniques known in the art, including mutagenesis. Polynucleotide variants may contain nucleotide substitutions, deletions, inversions and insertions.

PAP encoding nucleic acid molecules can be inserted into vectors and used as gene therapy vectors. In this context, a wide range of other host-vector systems suitable for the expression of PAP proteins or peptides thereof are known in the art, see for example, Sambrook et al., 1989, Current Protocols in Molecular Biology, 1995, supra. Gene therapy vectors can be delivered to a subject by, for example, intravenous injection, local administration (U.S. Pat. No. 5,328,470), implantation or by stereotactic injection (see e.g., Chen et al., PNAS 91:3054-3057 (1994)). Vectors for expression in mammalian hosts are disclosed in Wu et al. (1991) J. Biol. Chem. 266:14338; Wu and Wu (1988) J. Biol. Chem. 263:14621; and Zenke et al. (1990) Proc. Nat'l. Acad Sci. USA 87:3655. The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system. Preferred for use in the present invention are adenovirus vectors. These vectors may be employed to deliver and express a wide variety of genes, including, but not limited to cytokine genes such as those of the interferon gene family and the interleukin gene family. A preferred method for delivery of the expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct in host cells with complementary packaging functions and (b) to ultimately express a heterologous gene of interest that has been cloned therein.

The PAP polypeptides, PAP polypeptide variants, PAP peptides, PAP polynucleotides encoding said polypeptides, variants and fragments, and the PAP agents useful in the methods of the invention can be incorporated into pharmaceutical compositions suitable for administration into a mammal. The term “mammal” as used herein refers to any mammal classified as a mammal, including humans, nice, cows, horses, dogs and cats. In a preferred embodiment of the invention, the mammal is a human. Such compositions typically comprise at least one PAP polypeptide, PAP polypeptide variant, PAP polypeptide fragment, PAP polynucleotide encoding said polypeptide, variant or fragment, or a combination thereof, and a pharmaceutically acceptable carrier. Methods for formulating the PAP compounds of the invention for pharmaceutical administration are known to those of skill in the art. See, for example, Remington: The Science and Practice of Pharmacy, 19^{th} Edition, Gennaro (ed.) 1995, Mack Publishing Company, Easton, Pa.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions. A pharmaceutical composition of the, invention is formulated to be compatible with its intended route of administration.

The pharmaceutical compositions of the invention, comprising PAP polypeptides, PAP polypeptide variants, PAP polypeptide fragments, polynucleotides encoding said PAP polypeptides, variants and fragments, as well as PAP agents, as defined above, are administered in therapeutically effective amounts. The “therapeutically effective amount” refers to a nontoxic dosage level sufficient to induce a desired biological result (e.g. a diminution of the severity of the symptoms associated with a pathological condition such as pancreatitis, pancreatic dysfunction and/or PAP autoimnunity). Amounts for administration may vary based upon the desired activity, the diseased state of the mammal being treated, the dosage form, method of administration, patient factors such as age, sex, and severity of disease. It is recognized that a therapeutically effective amount is provided in a broad range of concentrations. Such range can be determined based on in vitro and/or in vivo assays.

Therapeutic compositions of the PAP can be prepared by mixing the desired PAP molecule having the appropriate degree of purity with optional pharmaceutically acceptable carriers, excipients, or stabilizers (Remington's Pharmaceutical Sciences, 16th edition, Osol, A. ed. (1980)), in the form of lyophilized formulations, aqueous solutions or aqueous suspensions. Acceptable carriers, excipients, or stabilizers are preferably nontoxic to recipients at the dosages and concentrations employed, and include buffers such as Tris, HEPES, PIPES, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; salt-forming counter-ions such as sodium; and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, colloidal silica, magnesium trisilicate, polyvinyl pyrrolidone, and cellulose-based substances. Carriers for topical or gel-based forms include polysaccharides such as sodium carboxymethylcellulose or methylcellulose, polyvinylpyrrolidone, polyacrylates, polyoxyethylene-polyoxypropylene-block polymers, polyethylene glycol, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations.

Further carriers include sustained release preparations such as semipermeable matrices of solid hydrophobic polymers containing, for example, the PAP polypeptide, which matrices are in the form of shaped articles, e.g., films, liposomes or microparticles. It will be apparent to those persons skilled in the art that certain carriers may be more preferable depending upon, for instance, the route of administration and concentration of PAP polypeptide being administered.

Solutions or suspensions used for administering PAP can include the following components: a sterile diluent such as water for injection, saline solution; fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose. In one embodiment, a pharmaceutical composition can be delivered via slow release formulation or matrix comprising PAP polypeptide or DNA constructs suitable for expression of PAP polypeptide into or around a site within the body.

A variety of specific formulations that are specifically designed for the administration of cytokines such as PAP are known in the art, with different formulation parameters effecting the function of the agent so administered. For example, formulations including pegylated cytokines that complexed with bioadhesive polymers suitable for topical administration to mucosal tissues are described in U.S. Pat. No. 6,165,509. As is known in the art, formulations can also be manipulated to modulate the timed release of the therapeutic agent. For example, U.S. Pat. No. 5,102,872 discloses the administration of a microencapsulated composition comprising a cytokine conjugated with a polyoxyethylene polymer, and mixed with a release-modulating amount of human serum albumin. In this formulation, the microcapsules are administered parenterally, and release an effective amount of conjugated cytokine continuously over a period of 14-30 days.

Yet another formulation known in the art to be useful to facilitate the delivery of polypeptides such as PAP involves the use of liposomes which can be used to improve cytokine pharmacokinetics, immunomodulatory activity and reduce toxicity. As is know in the art, liposomes are also useful in transducing cells with polynucleotides and are therefore useful in embodiments of the invention pertaining to the administration of PAP polypeptides. Illustrative liposome-mediated transfection formulations can be found, for example in Ausubel et al., Current Protocols in Molecular Biology, Wiley Interscience Publishers, (1995), units 9 and 16. In addition, a number of methods for enhancing transfection efficiency ate known in the art such as those described in Gebrekidan et al., AAPS PharmSciTech, 2000; 1(4) article 28 which describes the use of poly (D, L-lactide-co-glycolide) microspheres containing plasmid DNA for gene delivery. Such compositions can be used to formulate a controlled-release delivery system for DNA that can protect the DNA from DNAse degradation without loss of functional activity.

PAP can also be administered in the form of a variety of sustained-release preparations. For example, PAP may be delivered a site for slow release via encapsulation or carrier materials such as liposomes, or other drug “shells” such as albumin (Albunex by Molecular Biosystems), sugars (Levovist by Schering), gelatins, or lipids. Other suitable examples of sustained-release preparations for use with polypeptides including semipermeable matrices of solid hydrophobic polymers containing the polypeptide, which matrices are in the form of shaped articles, e.g., films, or microcapsules. Examples of sustained-release matrices include polyesters, hydrogels (e.g., poly(2-hydroxyethyl-methacrylate) as described by Langer et al., J. Biomed. Mater. Res., 15: 167-277 (1981) and Langer, Chem. Tech., 12: 98-105 (1982) or poly(vinylalcohol)), polylactides (U.S. Pat. No. 3,773,919, EP 58,481), copolymers of L-glutamic acid and gamma ethyl-L-glutamate (Sidman et al., Biopolymers, 22: 547-556 (1983)), non-degradable ethylene-vinyl acetate (Langer et al., supra), degradable lactic acid-glycolic acid copolymers such as the Lupron Depot (injectable microspheres composed of lactic acid-glycolic acid copolymer and leuprolide acetate), and poly-D-(−)-3-hydroxybutyric acid (EP 133,988).

The route of administration may vary depending on the desired effect and/or outcome. Generally for initiation of a PAP mediated response, introduction of the PAP at or near the desired site of response is utilized. Examples of routes of administration include parenteral, e.g., intravenous, intradermal, intramuscular, subcutaneous, oral, transdermal (topical), transmucosal (e.g. a nasal spray), and rectal administration. The PAP polypeptide may also be administered by perfusion techniques, such as isolated tissue perfusion, to exert local therapeutic effects. In a preferred embodiment, a continuous intraperitoneal infusion of the peptide formulation allow for easy access of peptides to the portal circulation and subsequently to the pancreas. Alternatively, the infusion could be performed either using CSI (continuous subcutaneous infusion) or through a special IV access port that is indwelling in the venous system leading to the pancreas.

Embodiments of the invention include methods employing continuous infusion systems. Continuous infusion systems are devices typically used for continuously administering a fluid to a patient parenterally for an extended period of time or for, intermittently administering a fluid to a patient parenterally over an extended period of time without having to establish a new site of administration each time the fluid is administered. The fluid contains a therapeutic agent or agents. The device comprises a reservoir for storing the fluid before it is infused, a pump, a catheter, or other tubing for connecting the reservoir to the administration site via the pump, and control elements to regulate the pump. The device may be constructed for implantation, usually subcutaneously. In such a case, the therapeutic agent reservoir will usually be adapted for percutaneous refilling. Obviously, when the device is implanted, the contents of the reservoir will be at body temperature, and subject to the patient's body motion.

Solutions or suspensions used for parenteral, intradermal, or subcutaneous application can include the following components: a sterile diluent such as water for injection, saline solution; fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffets such as acetates, citrates or phosphates and agents for the adjustment of tonicity such as sodium chloride or dextrose. Regimens of administration may vary. A single dose or multiple doses of the agent may be used. Such regimens can vary depending on the severity of the disease and the desired outcome. Following administration of a PAP polypeptide to the mammal, the mammal's physiological condition can be monitored in various ways well known to the skilled practitioner familiar with Type I diabetes, or alternatively by monitoring the effects of administration of PAP on the pancreatic environment (e.g. beta cell proliferation).

In another aspect of the invention, methods and formulations that include a PAP are disclosed for the delivery of drugs or other agents (e.g. imaging agents) to the pancreas for local or systemic therapies. The invention also includes methods and formulations to deliver PAP to the pancreas before or after delivery of a drug to enhance the efficacy of the drug, in an unaltered form as a depot for slow release of drugs, in unaltered form as a drug carrier, or in an altered form as a drug conjugate.

Effective dosages and schedules for administering the PAP polypeptides may be determined empirically (e.g. using the models disclosed herein), and making such determinations is within the skill in the art. Those skilled in the art will understand that the dosage of PAP polypeptide that can be administered will vary depending on, for example, the mammal which will receive the PAP polypeptide, the route of administration, the particular type of molecule used (e.g. polypeptide, polynucleotide etc.) used and other drugs being administered to the mammal.

The PAP polypeptide may also be administered to the mammal in combination with effective amounts of one or more other therapeutic agents. The one or more other therapeutic agents or therapies may include, but are not limited to GLP or a related peptide as well as immunomodulators such as FAS ligand. The amounts of the therapeutic agent depend, for example, on what type of drugs are used, the pathological condition being treated, and the scheduling and routes of administration but would generally be less than if each were used individually.

Following administration of a PAP polypeptide to the mammal, the mammal's physiological condition can be monitored in various ways well known to the skilled practitioner. The therapeutic effects of the PAP polypeptides of the invention can be examined in in vitro assays and using in vivo animal models. A variety of well known animal models can be used to further understand the role of the PAP in the development and pathogenesis of Type I diabetes, and to test the efficacy of the candidate therapeutic agents.

The pharmaceutical compositions can be included in a container, pack, or dispenser together with instructions for administration. That result can be reduction and/or alleviation of the signs, symptoms, or causes of a disease or any other desired alteration of a biological system. For example, in a further embodiment of the invention, there are provided articles of manufacture and kits containing materials useful for treating pathological conditions with PAP. The article of manufacture comprises a container with a label. Suitable containers include, for example, bottles, vials, and test tubes. The containers may be formed from a variety of materials such as glass or plastic. The container holds a composition having an active agent which is effective for treating pathological conditions such as Type I diabetes. The active agent in the composition is preferably PAP. The label on the container indicates that the composition is used for treating pathological conditions with PAP.

The kit of the invention comprises the container described above and a second container comprising a buffer. It may further include other materials desirable from a commercial and user standpoint, including other buffers, diluents, filters, needles, syringes, and package inserts with instructions for use.

D. Typical Embodiments of the Invention

A promising method for the treatment of Type I diabetes is the promotion of physiological mechanisms that facilitate the growth and or rehabilitation of the pancreas such as the promotion of pancreatic cell growth. In this regard, the 175 amino acid hamster homologue of PAP which is known as a islet neogenesis associated protein (INGAP), and is observed to have the ability-to regenerate the pancreatic beta cells in hamsters (see, e.g. U.S. Pat. No. 5,834,590). In addition, PAP is observed to bind strongly to extracellular matrix proteins such as lamanin-1 and fibronectin and is able to induce cellular adhesion (see, e.g. Christa et al., Am. J. Physiology 271(6): G993-G1002 (1996)). Without being bound by a particular theory, the disclosure in U.S. Pat. No. 5,834,590 provides evidence that PAP facilitates the growth of pancreatic cells by directly stimulating ductal cells to grow and proliferate. In addition, the disclosure in Christa et al., Am. J. Physiology 271(6): G993-G1002 (1996) provides evidence that PAP can facilitate the growth of cells by inducing the cellular adhesion of cells within the pancreatic architecture in a manner that counteracts the disruption of pancreatic architecture and spatial disorganization of pancreatic cells that is observed in diabetes (see, e.g. Gannon et al., Development 127(3): 2883-2895 (2000); Yamagata et al., Diabetes 51(1): 114-123 (2002); and Zulewski et al., Diabetes 50(3): 521-533 (2001)).

As discussed herein, the hamster INGAP gene has homology to a 175 amino acid human protein designated pancreatitis-associated polypeptide (PAP, see, e.g. Orelle et al., J. Clin. Invest. 90, 2284-2291 (1992) and Lasserre et al., Cancer Res. 52, 5089-5095 (1992)). As further discussed herein, the 175 amino acid PAP protein is known to be a T-cell autoantigen that is recognized in a model of autoimmune diabetes (see, Gurr et al., Diabetes 51: 339-346, 2002). In these contexts, the methods disclosed herein are directed to methods including the use of a biologically active fragment of PAP to stimulate beta cell growth and at the same time to both avoid and/or overcome the T-cell mediated autoimmune attack on the pancreas.

An illustrative embodiment of the invention is a method of using a biologically active fragment of pancreatitis associated protein to facilitate the growth of a pancreatic cell in a mammal in a manner that simultaneously minimizes the immunostimulation of a leukocyte produced by the mammal that immunospecifically recognizes an epitope present on the full length pancreatitis associated protein. In particular, the method involves administering to the mammal a biologically active fragment of pancreatitis associated protein comprising the amino acid sequence IGLHDPTQGTEPNGE (SEQ ID NO: 3). In this method, the mammal receiving this biologically active fragment of pancreatitis associated protein is selected to have a leukocyte that immunospecifically recognizes an epitope present on the homologous pancreatitis associated protein comprising the sequence MLPPMALPSVSWMLLSCLMLLSQVQGEEPQRELPSARIRCPKGSIKAYGSHCYALFLSPKSWT DADLACQKRPSGNLVSVLSGAEGSFVSSLVKSIGNSYSYVWIGLHDPTQGTEPNGEGWEWS SSDVMNYFAWERNPSTISSPGHCASLSRSTAFLRWKDYNCNVRLPYVCKFTD (SEQ ID NO: 1) that is not present on the biologically active fragment of pancreatitis associated protein. Consequently, the administration of the biologically active fragment of pancreatitis associated protein facilitates the growth of the pancreatic cell but does not immunospecifically stimulate the leukocyte to the same degree as would the administration of an equivalent amount of the homologous pancreatitis associated protein.

In this method, the biologically active fragment of the pancreatitis associated protein can facilitate the growth of the pancreatic cell directly, for example by stimulating the proliferation of a specific pancreatic cell (e.g. a beta cell). Alternatively, the biologically active fragment of the pancreatitis associated protein can facilitate the growth of the pancreatic cell indirectly, for example by contributing to the formation of a substrate conducive to pancreatic cell growth (e.g. an extracellular matrix substrate). The biological activity of the biologically active fragment of pancreatitis associated protein comprising the amino acid sequence IGLHDPTQGTEPNGE (SEQ ID NO: 3) can be measured by a variety of assays known in the art, for example the proliferation and adhesion assays disclosed in the Examples.

In this method, the leukocyte that immunospecifically recognizes an epitope present on the homologous pancreatitis associated protein is typically a B lymphocyte or a T lymphocyte. The immunospecificity of such leukocytes can be measured by any one of a wide variety of methods known in the art, for example Western blotting or enzyme linked immunoadsorbent assays (for B lymphocytes) or T lymphocyte proliferation assays. These assays are typically used to determine whether a specific biologically active fragment of pancreatitis associated protein does not imunospecifically stimulate the leukocyte to the same degree as would the administration of an equivalent amount of the homologous pancreatitis associated protein. In this context, the term “equivalent amount” refers to an equal molar amount of individual molecules (and therefore epitopes) that are available for recognition by the leukocyte (and not absolute protein concentration).

Another typical embodiment of the invention is a method of inhibiting the onset of Type I diabetes in a mammalian subject predisposed to Type I diabetes comprising administering to the subject a therapeutically effective amount of a PAP peptide comprising the amino acid sequence shown in SEQ ID NO: 3. In such methods the PAP peptide can be administered via any one of the variety of methods known in the art. In one embodiment of the invention, the PAP peptide is administered to the subject via a continuous infusion pump. Embodiments of the invention include those wherein the mammalian subject has at least one T lymphocyte that is reactive to the 175 amino acid PAP protein shown in SEQ ID NO: 1, yet is not reactive to the PAP peptide shown in SEQ ID NO: 3. Preferably the peptide formulation includes a pharmaceutically acceptable carrier such a physiologic saline. Optionally the PAP peptide is conjugated to a polyol. Related embodiments of the invention include administering a second agent (including those typically used in the treatment of diabetes) such as one selected from the group consisting of human insulin, human glucagon-like peptide 1 and Fas ligand. While the methods disclosed herein are directed primarily to the treatment of type I diabetes, the use of such methods in the prevention and treatment of other diseases associated with pancreatic dysfunction are also contemplated. In addition, such methods can be used in certain contexts involving Type II diabetes.

A related embodiment of the invention is a method of stimulating neogenesis of pancreatic beta cells in an individual having a predisposition to Type I diabetes comprising administering to the subject a PAP peptide comprising the amino acid sequence shown in SEQ ID NO: 3. A predisposition to diabetes can be measured by any one of the wide variety of methods known in the art. In a typical embodiment, the predisposition to Type I diabetes is determined by the presence of islet cell autoantibodies or insulin autoantibodies. In a specific method, the islet cell autoantibody recognizes islet cell antigen 512 (ICA 512). Alternatively, the predisposition to Type I diabetes is determined by the presence of T cells that recognize an epitope on the 175 amino acid PAP protein shown in SEQ ID NO: 1.

Yet another embodiment of the invention is a method of inhibiting the onset of Type I diabetes in a mammalian subject predisposed to Type I diabetes comprising administering to the subject a therapeutically effective amount of a PAP peptide comprising the amino acid sequence shown in SEQ ID NO: 3, wherein the PAP peptide does not possess an epitope present in the PAP protein comprising the amino acid sequence shown in SEQ ID NO: 1 that is recognized by autoimmune T-cells in the individual.

Yet another embodiment of the invention is a method of stimulating neogenesis of pancreatic beta cells in an individual having a predisposition to Type I diabetes comprising determining whether the mammalian subject possesses a T lymphocyte that is reactive to the 175 amino acid PAP protein shown in SEQ ID NO: 1, is not reactive to the PAP peptide shown in SEQ ID NO: 3, and then administering to the subject a therapeutically effective amount of a PAP peptide comprising the amino acid sequence shown in SEQ ID NO: 3 to the subject.

Yet another embodiment of the invention is a method of inhibiting the expression in a mammalian subject of an epitope present on the 175 amino acid PAP protein shown in SEQ ID NO: 1, wherein the epitope is recognized by an autoimmune T cell in Type I diabetes comprising exposing a pancreatic ductal cell in the subject to an amount of a PAP peptide comprising the amino acid sequence shown in SEQ ID NO: 3 sufficient to stimulate beta cell neogenesis. Optionally, the expression of the 175 amino acid PAP protein shown in SEQ ID NO: 1 is measured via a northern blot analysis or polymerase chain reaction analysis. In measuring the expression of the 175 amino acid PAP protein shown in SEQ ID NO: 1, the expression of a control gene such as actin can be used in comparative analyses of expression. Optionally, the proliferative response of the pancreatic ductal cell is measured via [³H]TdR incorporation.

In such methods the subject typically exhibits at least one factor associated with a predisposition to Type I diabetes. Preferably the factor associated with a predisposition to Type I diabetes is determined is the presence of islet cell autoantibodies or insulin autoantibodies. In a preferred embodiment, the factor associated with a predisposition to Type I diabetes is determined is the presence of an autoimmune T-cell that recognizes an epitope present on the 175 amino acid PAP protein shown in SEQ ID NO: 1. Alternatively the factor associated with a predisposition to Type I diabetes is determined is the presence of an autoantibody that recognizes islet cell antigen 512 (ICA 512).

Yet another embodiment of the invention is a composition comprising a PAP peptide having the amino acid sequence shown in SEQ ID NO: 3 and a pharmaceutically acceptable carrier. Optionally the peptide is conjugated to a polyol. In one embodiment, such composition may be included in an article of manufacture or kit. The composition may be a pharmaceutically acceptable formulation useful, for instance, in inducing or stimulating neogenesis in mammalian beta cells or for treating an immune related disorder, such as diabetes. In one embodiment of the invention, the PAP peptide is a peptide comprising amino acids 104-118 and having an amino acid substitution at one or more amino acid residues of residues 104-118.

Embodiments of the invention also include methods which reduce the amount of PAP autoantigen that is presented to immune system. Such methods inhibit the ability of PAP reactive immune cells (e.g. cytotoxic T-cells and antibody producing B cells) to arise, develop and mature by reducing the amount of circulating antigen capable of stimulating these immune cells. Such methods stimulate beta cell neogenesis using a biologically active fragment of PAP rather than the complete 175 amino acid PAP protein, a protein which is a known T-cell autoantigen. As a peptide having only a small portion of the 175 amino acid PAP protein autoantigen (e.g. the 15 amino acid peptide sequence shown in SEQ ID NO: 3 which contains less than 10% of the PAP amino acid sequence) will not include the complete complement of epitopes present 175 amino acid PAP protein autoantigen, those autoimmune epitopes that are missing or can no longer adopt an immunogenic secondary structure are therefore no longer capable of stimulating an immune response. Consequently, methods which use such small peptides to facilitate pancreatic cell growth (e.g. by stimulating beta cell neogenesis) instead of 175 amino acid PAP protein autoantigen serve to repopulate a cell lineage that is lost in Type I diabetes while at the same time, avoiding the stimulation of an autoimmune reaction in a pathology characterized by autoimmunity. As described herein, the stimulation of autoreactive T-cell populations can be measured by methods known in the art (see, e.g. Gurr et al., Diabetes 51: 339-346, 2002). In the preferred embodiments of the invention, the ability of a PAP peptide to stimulate autoreactive T-cell populations is less than stimulation of autoreactive T-cell populations that is observed using a PAP polypeptide having at least about 130 amino acids of protein shown in SEQ ID NO 1.

As studies indicate that PAP is involved in autocrine growth, in contexts where the consequences of PAP biological activity ultimately decrease the amount of circulating PAP (as typically occurs in autocrine growth regulation), the use of such peptides provides methods of decreasing the amount of circulating PAP autoantigen. Typical embodiments include a method of decreasing the amount of PAP antigen capable of stimulating an autoimmune response by administering an amount of PAP peptide sufficient to stimulate beta cell neogenesis. Preferably the PAP antigen is the peptide shown in SEQ ID NO: 3.

In addition, it has long been accepted that the response of T cells to a protein antigen is strongly influenced by parameters governing processing and presentation of the immunodominant epitopes. Recent evidence has suggested, however, that subtle changes in the nature of the ligand itself may also affect the outcome of T-cell receptor (TCR) ligation, necessitating a re-evaluation of previously accepted paradigms of T-cell activation. In particular, activation may no longer be regarded as an all-or-nothing event, but appears to involve both quantitative and qualitative dimensions. This revolution in understanding has emanated from the recent discovery that analogues of immunogenic peptides, so-called altered peptide ligands (APLs), may elicit a subset of normal activation events, or profoundly influence responses to the wild-type epitope. As such, the potential offered by APLs for modifying the outcome of deleterious immune responses involved in autoimmunity has not passed unnoticed. Indeed, the design and exploitation of novel reagents based on the structure of autoantigenic epitopes has enjoyed some measure of success in the treatment of experimental models of autoimmune disease and holds promise for their exploitation within the clinical arena. Fairchild. Eur J Immunogenet April 1997;24(2):155-67.

Consequently, such advances in knowledge of how T cells see antigens provide for improved therapeutic strategies using the PAP peptide (see also Wraith, Int Arch Allergy Immunol December 1995;108(4):355-9). Specifically, T lymphocytes recognize processed forms of antigen which can be mimicked by synthetic peptides designed and built in the laboratory. It is clear from recent work that these synthetic peptides, when given systemically in solution, induce a state of hyporesponsiveness in naive T cells thereby specifically preventing a subsequent immune response. Moreover systemic administration of soluble peptides can inhibit ongoing immune responses. Taken together this new information offers great promise for future development of PAP antigen-based drugs for the treatment of autoimmune diabetes. Typical embodiments include a method of inducing a state of PAP hyporesponsiveness in naive T cells (thereby specifically preventing a subsequent immune response to PAP antigen) by administering an amount of PAP peptide sufficient to stimulate beta cell neogenesis.

The literature suggests that one can finely tuned such methods to allow for both ongoing autoimmune cellular destruction and to avoid over-stimulation of the Islets. One approach is to create an immunoprivileged site in the pancreas during PAP infusion. The immuno-privileged site can be obtained in two fundamental manners. The most expedient is to co-infuse FAS-ligand concomitantly with the PAP peptide. It is well known that Sertoli cells create an immuno-privileged site, predominantly due to the presence of FAS ligand (FAS-L) on the surface of the cells. Fas-L has also been used to protect transplanted islets from destruction without the use of concomitant immunosuppressive agents. The use of soluble Fas-L together with the PAP peptide will promote neogenesis of islets while conferring immuno-privilege during the neogenesis. This immuno-privilege will allow the newly generated cells to remain protected from T cell mediated autoimmunity during their growth.

A second method for creating immunoprivilege of islets during neogenesis is to create a protein complex that contains both Fas-L and PAP. This can be easily accomplished by covalently attaching both the Fas-L and the PAP at either ends of a synthetic soluble sugar or carbohydrate. For example, using streptavidin/biotin binding, a complex that has a linear sugar molecule with Fas-L at one end and PAP at the other would be relatively simple to produce. This complex can maintain the ability of PAP to bind to its receptor while maintaining Fas-L immunoprivilege at the other end.

As disclosed herein, because PAP stimulates beta cell neogenesis and because the 175 amino acid PAP protein contains epitopes recognized by autoreactive T-cell in a diabetes model, the assays disclosed herein (e.g. those described in Examples 1 and 2 below) can be used in cell based assays designed to assess the effects of compounds which can modulate the beta cell neogenic and/or T-cell immunogenic properties of PAP polypeptides. For example the disclosure provided herein allows one to examine the effects that test PAP peptide and or a related compound has on beta cell neogenesis and/or T-cell autoimmune reactivity.

Such cell based assays can also be used for example to evaluate how various PAP formulations impact specific physiological processes in diabetes such as the autoimmune phenomena observed in this pathology. Related embodiments of the invention are cell based assays which can be used to dissect specific physiological aspects of pathological conditions such as diabetes. Related embodiments of the invention are cell based assays which can be used to evaluate how additional agents may effect such processes. One such embodiment of the invention is a method of identifying an agent capable of modulating beta cell neogenesis by exposing a ductal pancreatic cell to an amount of PAP and, alternatively or in addition, exposing the ductal pancreatic cell to a small molecule or polypeptide agent and observing the agent's effect on beta cell neogenesis. A related embodiment of the invention is a method of identifying an agent capable of modulating T cell immunoreactivity to a peptide having the biological activity of the protein shown in SEQ ID NO: 1 by exposing a mammalian subject to an amount of PAP protein shown in SEQ ID NO: 1 and additionally exposing the mammalian subject to a small molecule or polypeptide agent and observing the agent's effect on T-cell immunoreactivity to PAP protein shown in SEQ ID NO: 1.

The cell based assay methods described herein can be employed in a number of contexts. For example the methods described above can be practiced serially as the effects of compounds that have the ability modulate beta cell neogenesis examined. In one such embodiment of the invention, beta cell neogenesis or autoimmunity in response to PAP is first examined to determine the effects of PAP in that specific context. A variation of the method can then be repeated using a test compound in place of PAP and the effects on beta cell neogenesis and/or autoimmunity in response to the test compound in the model then being examined to identify molecules which can produce physiological effects that are similar or dissimilar to PAP. In a related embodiment PAP and a test compound can be added simultaneously to see if the test compound can modulate the effects of PAP in a manner that may have some clinical applicability, for example to modulate beta cell neogenesis and/or autoimnunity with fewer side effects etc.

Throughout this application various articles, patents, patent applications, GenBank accession numbers (which, as is known in the art provide a reference of sequence and publication information), and other publications etc. are referenced (e.g. WO 00/38706). The disclosures of these publications etc. are hereby incorporated by reference herein in their entireties. Certain portions of these incorporated publications etc. may be disclosed for purposes of providing a full, clear and concise written description of the various aspects of the embodiments of the invention.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

TABLE 1
Human HIP/PAP

| | |
|---|---|
| A. 175 amino acid complete sequence | (SEQ ID NO: 1) |

MLPPMALPSVSWMLLSCLMLLSQVQGEEPQRELPSARTRCPKGSKAYGSHCYALFLSPKSWTDADLACQKRPSGNLVSV
LSGAEGSFVSSLVKSIGNSYSYVWIGLHDPTQGTEPNGEGWEWSSSDVMNYFAWERNPSTISSPGHCASLSRSTAFLRW
KDYNCNVRLPYVCKFTD

| | |
|---|---|
| B. 25 amino acid Biologically active peptide | (SEQ ID NO: 2) |
| YVWIGLHDPTQGTEPNGEGWEWSSSD | |
| C. 15 amino acid Biologically active peptide | (SEQ ID NO: 3) |
| IGLHDPTQGTEPNGE | |
| D. Native Nucleotide sequence | (SEQ ID NO: 4) |

GGGAGGGTCCCTTCCTCAGGGAGCACAGGAACTCTGAGACTCAGCAAGGGTGTCCTGGGAGGGCTCGGGGATGGGAGAG
TACACAGATTCACAACTCATTCAGAACTGTAGAAGATGATGGATGTGACCAAGATCACTTTAGTCCTAGGGGACTAGAG
AAGGAAAATGACATGAGGCAGTGGGGTATCTGTGTGTTCTCCCACTGACCACGCTTTCTTTAGTGACTCCTGATTGCCT
CCTCAAGTCGCAGACACTATGCTGCCTCCCATGGCCCTGCCCAGTGTATCTTGGATGCTGCTTTCCTGCCTCATGCTGC
TGTCTCAGGTTCAAGGTGAAGAACCCCAGAGGGAACTGCCCTCTGCACGGATCCGCTGTCCCAAAGGCTCCAAGGCCTA
TGGCTCCCACTGCTATGCCTTGTTTTTGTCACCAAAATCCTGGACAGATGCAGATCTGGCCTGCCAGAAGCGGCCCTCT
GGAAACCTGGTGTCTGTGCTCAGTGGGGCTGAGGGATCCTTCGTGTCCTCCCTGGTGAAGAGCATTGGTAACAGCTACT
CATACGTCTGGATTGGGCTCCATGACCCCACACAGGGCACCGAGCCCAATGGAGAAGGTTGGGAGTGGAGTAGCAGTGA
TGTGATGAATTACTTTGCATGGGAGAGAAATCCCTCCACCATCTCAAGCCCCGGCCACTGTGCGAGCCTGTCGAGAAGC
ACAGCATTTCTGAGGTGGAAAGATTATAACTGTAATGTGAGGTTACCCTATGTCTGCAAGTTCACTGACTAGTGCAGGA
GGGAAGTCAGCAGCCTGTGTTTGGTGTGCAACTCATCATGGGCATGAGACCAGTGTGAGGACTCACCCTGGAAGAGAAT
ATTCGCTTAATTCCCCCAACCTGACCACCTCATTCTTATCTTTCTTCTGTTTCTTCCTCCCCGCTGTCATTTCAGTCTC
TTCATTTTGTCATACGGCCTAAGGCTTTAAAGAGCAATAAAATTTTTAGTCTGC

### EXAMPLES

### Example 1

### [³H]TdR Examination of the Proliferative Response of Pancreatic Cells to PAP Peptide

[³H]TdR Incorporation.

Examinations of PAP activity can be carried out by methods known in the art (see, e.g. Rafaeloff et al., J. Clin. Invest. 1997, 99:2100-2109). For example, a IGLHDPTQGTEPNGE (SEQ ID NO: 3) pentadecapeptide corresponding to amino-acids 104-118 of hu-PAP/HIP can be synthesized commercially by a company such as Genosys Biotechnologies the Woodlands, Tex.). The proliferative response of pancreatic duct cells and/or cell lines (e.g. ARIP and HIT-T15) to PAP peptide can then be quantified by [³H]TdR incorporation. Cells can be plated onto 6-well plates and cultured in Ham's F-12K media containing 10% FBS for 24-48 h or until reaching 50-60% confluency. The media can then be removed and the cells can be incubated for 24 hours either with varying concentrations of PAP peptide (100-1500 ng/ml) or an equal volume of control diluent in the presence of 10 μCi of [³H]TdR (80.4 Ci/mmol, DuPont-New England Nuclear). As a negative control one can use a cytokine, for example 60-500 ng/ml nerve growth factor (NGF) protein in the incubation instead of PAP. DNA can be precipitated with 15% TCA and extracted with 2 N NaOH and the recovered radioactivity then measured. Data can be recorded as recovered cpm/μg DNA and analyzed using ANOVA and post hoc Dunnett's test. Significance can be accepted at the 5% level.

### Example 2

### Examination of the Proliferative Response of Pancreatic Cells to PAP Peptide

Animal Experiments

Male Wistar rats (200-220 g of weight) can be 90% depancreatized and maintained on standard rat chow. Recombinant PAP or synthetic PAP peptide can be injected i.p. at a dose of 1 mg/kg of body weight into 20 rats every day. Control rats can be injected i.p. with 50 mM of control buffer without PAP polypeptide after the 90% pancreatectomy. In both groups the injections can be continued until the 60th postoperative day. Rats can be fasted overnight before blood sampling, and the plasma glucose level can be measured by the glucose oxidase method, using Glucose Auto and Stat GA-1110 (Kyoto DaiicHi Instrument, Kyoto). The statistical significance of differences between rats injected with PAP polypeptide and those injected with control buffer can be analyzed by using Student's t test. The residual pancreatic tissues can be removed 2 months after the partial pancreatectomy and fixed in Bouins' solution. Hydrated 5-lim sections of paraffin-embedded pancreatic tissues can be stained for insulin by the labeled streptavidin biotin method, using an LSAB kit (Dakopatts, Glostrup, Denmark). After being stained, the relative volumes of beta cells can be measured by the point-counting method.

In Vitro Experiment

Pancreatic islets can be isolated from male Wistar rats (body weight 200-250 g) by using density separation on a dextran gradient and cultured free-floating in RPMI 1640 medium/10% fetal calf serum/penicillin G at 100 jig/ml/streptomycin at 100 ug/mil for 48 hr to allow recovery from the isolation procedure. After this initial period, islets can be transferred to 24-well culture dishes in groups of 50 islets. The islets can be cultured in RPMI 1640 medium/2.7 MM n-glucose/2% fetal calf serum/penicillin G at 100 4 ml/streptomycin at 100 ug/ml in the presence of increased concentrations of PAP polypeptide for 72 hr. During the last 24 hr, the islets can be cultured in the above medium to which [methyl-3H]thymidine at 10 uCi/ml (Amersham; 1 Ci=37 GBq) had been added. To estimate the amount of [3H]thymidine incorporated into newly synthesized DNA, the islets can be washed after the culture period and sonicated in 10 mM Tris-HCl/5 mM EDTA. DNA can be precipitated by the addition of ⁷% ice-cold trichloroacetic acid and trapped by filtration on a glass-fiber disc (Whatman GF/C). The discs can be dried, and radioactivity can be counted after the addition of scintillation fluid (Packard Ultima Gold F). The DNA content of the islets can be measured by a fluorometric DNA assay using Hoechst 33258. To estimate labeling indices for insulin-positive cells, the [H] thymidine labeled islets can be fixed in 4% paraformaldehyde for 20 min and embedded in OCT compound. Ten-micrometer sections can be cut with a cryostat and immunostained with antiporcine insulin guinea pig antiserum (DAKO, 1:500) and Vectastain ABC-GO kit (Vector Laboratories). Autoradiography of the immunostained sections can be performed by using Konica NR-M2 emulsion.

### Example 3

### Examination of the Adhesion Response of Cells to PAP Peptides

The following assays can be used to examine the ability of various PAP peptides to promote cellular adhesion and to bind to extracellular matrices. While hepatocytes are used in the specific PAP assay procedures that are described below per the disclosure in Christa et al., Am. J. Physiology 271(6): G993-G1002 (1996), artisans understand that the use of analogous assays using pancreatic cells are within the level of skill in this art.

Rat hepaloyte adhesion on HIP/PAP protein. Hepatocytes can be isolated from male Sprague-Dawley rats using procedures known in the art such as the two-step collagenase procedure. This two-step collagenase procedure technique has been used successfully by several laboratories for analyzing the interactions of hepatocytes with various substrates: when hepatocytes are used immediately after their isolation in serum-free medium, they are almost devoid of extracellular matrix. Experiments can be conducted using serum-free medium (75% minimum essential medium, 25% medium 199, and 0.02% bovine serum albumin) to obviate potential competitive inhibition by serum glycoptoteins. Tissue culture plates (96-well flat bottom) can be precoated overnight at +4° C. and for 2 h at 37° C. with one of the substrates in 100 μl serum-free medium: 1) 20 μg/ml of lamin-1 from the murine Engelbreth-Holm-Swarm tumor (16), 2) 20 μg/ml of type I collagen from rat tails, and 3) 0.1-4 μg/well of E. coli-purified recombinant pre-HIP/PAP or CRD. Then 100 μl of serum-free medium containing 3% bovine serum albumin (BSA) can be added to block the remaining protein binding sites and then incubated for 30 min. The medium can be removed, and hepatocytes (6×10⁴ cells/well in 100 μl) can be added in a serum-free medium containing 0.2% BSA, with or without a 15-min preincubation with pre-HIP/PAP or CRD. After incubation for 30 min, the supernatants can be transferred to other wells and any attached hepatocytes can be rinsed twice with PBS. The percentage of adherent cells can be determined by lactate dehydrogenase activity in the supernatants and cell layers. Adhesions can be performed in duplicate in three independent experiments.

Enzyme-linked immunosorbent assay test to measure HIP/PAP binding on extracellular matrix protein. Flat-bottomed, 96-well enzyme-linked immunosorbent assay (ELISA) plates can be precoated with different substrates: 0.5 μg in 100 μl of 50 mM sodium carbonate buffer, pH 9.6, overnight at +4° C., lamnin-1 (Sigma) (15), type I and IV collagen (Harbor Bio-products), human fibronectin (Boehringer), heparansulfate-proteoglycan from the murine Engelbreth-Holn-Swarm tumor (Becton Dickinson), and BSA (Sigma). Nonspecific binding sites can be blocked by PBS/0.2% Tween 20 for 1 h at 37° C. Pre-HIP/PAP and its CRD (from 0.04 to 2 μg n 100 μl PBS/0.05% Tween 20) can be added for 2 h at 37° C. The wells can be washed with PBS/0.05% Tween 20, incubated with total HIP/PAP IgGs (1:5,000; 2 h at 37° C.), washed again, and finally incubated with alkaline phosphatase-conjugated mouse anti-rabbit monoclonal secondary antibodies (1:20,000, 1 h at 37° C.). Detection can be performed and absorbance can be measured at 405 nm.

1. A method of using a pancreatitis associated peptide to facilitate the growth of a pancreatic cell in a mammal in a manner that simultaneously minimizes the immunostimulation of a leukocyte produced by the mammal that immunospecifically recognizes an epitope present on a pancreatitis associated protein having homology to the pancreatitis associated peptide, the method comprising:
(a) administering to the mammal a pancreatitis associated peptide comprising the amino acid sequence IGLHDPTQGTEPNGE (SEQ ID NO: 3), wherein the pancreatitis associated peptide can facilitate the growth of the pancreatic cell; wherein
(b) the mammal produces a leukocyte that immunospecifically recognizes an epitope present on the homologous pancreatitis associated protein comprising the sequence MLPPMALPSVSWMLLSCLMLLSQVQGEEPQRELPSARIRCPKGSKAYGSHCYALFLSPKSWT DADLACQKRPSGNLVSVLSGAEGSFVSSLVKSIGNSYSYVWIGLHDPTQGTEPNGEGWEWS SSDVMNYFAWERNPSTISSPGHCASLSRSTAFLRWKDYNCNVRLPYVCIFTD (SEQ ID NO: 1), wherein the epitope is not present on the pancreatitis associated peptide of step (a),
so that the administration of the pancreatitis associated peptide facilitates the growth of the pancreatic cell but does not immunospecifically stimulate the leukocyte to the same degree as an equivalent amount of the homologous pancreatitis associated protein.

2. The method of claim 1, wherein the leukocyte that immunospecifically reacts to an epitope present on a homologous pancreatitis associated protein is a B lymphocyte.

3. The method of claim 2, wherein the immunoreactivity of the B lymphocyte can be assayed using Western blotting or an enzyme linked immunoadsorbent assay (ELISA).

4. The method of claim 1, wherein the leukocyte that immunospecifically reacts to an epitope present on a homologous pancreatitis associated protein is a T lymphocyte.

5. The method of claim 4; wherein the immunoreactivity of the T lymphocyte can be assayed using a T lymphocyte proliferation assay.

6. The method of claim 1, wherein the pancreatitis associated peptide ability to facilitate pancreatic cell growth can be measured in a [³H]TdR incorporation cell proliferation assay or a cellular adhesion assay.

7. The method of claim 6, wherein the mammal is a human individual identified as having a predisposition to Type I diabetes.

8. The method of claim 7, wherein the predisposition to Type I diabetes is determined by the presence of islet cell autoantibodies or insulin autoantibodies.

9. The method of claim 1, wherein the pancreatitis associated peptide comprising the amino acid sequence IGLHDPTQGTEPNGE (SEQ ID NO: 3) is linked to a heterologous polypeptide sequence.

10. The method of claim 1, wherein the pancreatitis associated peptide is conjugated to a polyol.

11. A method of stimulating neogenesis of pancreatic beta cells in an individual having a predisposition to Type I diabetes comprising:
(a) determining whether the mammalian subject possesses a T lymphocyte that is:
(i) reactive to the 175 amino acid pancreatitis associated polypeptide protein shown in SEQ ID NO: 1; and
(ii) not reactive to the pancreatitis associated polypeptide peptide shown in SEQ ID NO: 3; and
(b) administering to the subject a therapeutically effective amount of a pancreatitis associated peptide comprising the amino acid sequence shown in SEQ ID NO: 3.

12. A method of inhibiting the expression in a mammalian subject of an epitope present on the 175 amino acid pancreatitis associated polypeptide protein shown in SEQ ID NO: 1, wherein the epitope is recognized by an autoimmune T cell in Type I diabetes, the method comprising exposing a pancreatic ductal cell in the subject to an amount of a pancreatitis associated polypeptide peptide comprising the amino acid sequence shown in SEQ ID NO: 3 sufficient to stimulate beta cell neogenesis.

13. The method according to claim 12, wherein the expression of the 175 amino acid pancreatitis associated polypeptide protein shown in SEQ ID NO: 1 is measured via a northern blot analysis or polymerase chain reaction analysis.

14. The method of claim 12, wherein a pancreatitis associated polypeptide peptide mediated proliferative response of the pancreatic ductal cell is measured via [³H]TdR incorporation.

15. The method of claim 12, wherein the subject exhibits at least one factor associated with a predisposition to Type I diabetes.

16. The method of claim 15, wherein the factor associated with a predisposition to Type I diabetes is determined is the presence of islet cell autoantibodies or insulin autoantibodies.

17. The method of claim 16, wherein the factor associated with a predisposition to Type I diabetes is determined is the presence of an autoantibody that recognizes islet cell antigen 512 (ICA 512).

18. The method of claim 15, wherein the factor associated with a predisposition to Type I diabetes is determined is the presence of an autoimmune T-cell that recognizes an epitope present on the 175 amino acid pancreatitis associated polypeptide protein shown in SEQ ID NO: 1.

19. The method of claim 12, wherein the peptide is conjugated to a polyol.

20. A method of inhibiting the onset of Type I diabetes in a mammalian subject predisposed to Type I diabetes comprising administering to the subject a therapeutically effective amount of a pancreatitis associated polypeptide peptide comprising the amino acid sequence shown in SEQ ID NO: 3, wherein the mammalian subject is selected to have at least one leukocyte that is:
(a) immunospecifically reactive to the 175 amino acid pancreatitis associated polypeptide protein shown in SEQ ID NO: 1; and
(b) not immunospecifically reactive to the pancreatitis associated polypeptide peptide shown in SEQ ID NO: 3.

21. The method of claim 20, wherein the pancreatitis associated polypeptide peptide is administered to the subject via a continuous infusion pump.

22. The method of claim 20, further comprising administering a second agent selected from the group consisting of human insulin, human glucagon-like peptide 1 and Fas ligand.

23. The method of claim 20, wherein the immunospecific reactivity of the leukocyte can be assayed via a T lymphocyte proliferation assay.

24. The method of claim 20, wherein the pancreatitis associated polypeptide peptide is conjugated to a polyol.

25. A method of stimulating neogenesis of pancreatic beta cells in an individual having a predisposition to Type I diabetes comprising administering to the subject a pancreatitis associated polypeptide peptide comprising the amino acid sequence shown in SEQ ID NO: 3.

26. The method of claim 25, wherein the predisposition to Type I diabetes is determined by the presence of islet cell autoantibodies or insulin autoantibodies.

27. The method of claim 26, wherein the islet cell autoantibody recognizes islet cell antigen 512 (ICA 512).

28. The method of claim 25, wherein the predisposition to Type I diabetes is determined by the presence of T cells that recognize an epitope on the 175 amino acid pancreatitis associated polypeptide shown in SEQ ID NO: 1.

29. A method of inhibiting the onset of Type I diabetes in a mammalian subject predisposed to Type I diabetes comprising administering to the subject a therapeutically effective amount of a pancreatitis associated polypeptide peptide comprising the amino acid sequence shown in SEQ ID NO: 3, wherein the pancreatitis associated polypeptide peptide does not possess an epitope present in the pancreatitis associated polypeptide protein comprising the amino acid sequence shown in SEQ ID NO: 1 that is recognized by autoimmune T-cells in the individual.

30. A composition comprising a pancreatitis associated polypeptide peptide having the amino acid sequence shown in SEQ ID NO: 3 and a pharmaceutically acceptable carrier.

31. A composition comprising a pancreatitis associated polypeptide peptide having the amino acid sequence shown in SEQ ID NO: 3, wherein the peptide has an amino acid substitution at I104, G105, L106, H107, D108, P109, T110, Q111, G112, T113, E114, P115, N116, G117 or E118.
